(19) United States
(12) Patent Application Publication
(10) Pub. No.: US 2025/0186401 A1
(43) Pub. Date: Jun. 12, 2025

(54) COMBINATORIAL ACTIVATION OF GABA(B) AND A2 ADRENERGIC RECEPTORS PROVIDES RELIEF FROM STRESS INDUCED DEPRESSION

(71) Applicant: University of Florida Research Foundation, Incorporated, Gainesville, FL (US)

(72) Inventors: Kirill Martemyanov, Gainesville, FL (US); Laurie Sutton, Gainesville, FL (US)

(21) Appl. No.: 18/842,118

(22) PCT Filed: Mar. 15, 2023
(86) PCT No.: PCT/US2023/064443

§ 371 Date: Aug. 28, 2024

Related U.S. Application Data
(60) Provisional application No. 63/320,588, filed on Mar. 16, 2022.

(51) Int. Cl. A61K 31/4168; A61K 31/197; A61P 25/24

(52) U.S. Cl. CPC A61K 31/4168; A61K 31/197; A61P 25/24

(57) ABSTRACT

Described are compositions, combinations, and methods of using the compositions and combinations for use in treating depression. The compositions and combinations comprise a GABA B receptor agonist and an alpha-2 adrenergic receptor agonist.

### CROSS-REFERENCE TO RELATED APPLICATION

This application claims the benefit of priority of U.S. provisional application No. 63/320,588, which was filed on Mar. 16, 2022, and which is incorporated herein by reference.

### STATEMENT REGARDING FEDERALLY SPONSORED RESEARCH OR DEVELOPMENT

This invention was made with government support under grant number R01 MH105482, awarded by the National Institutes of Health. The government has certain rights in the invention.

### BACKGROUND

Major depressive disorder is a major health problem not currently adequately controlled by prescription medications. Most available drugs on the market have inadequate efficacy, take a long time to exert effects, and/or and help only a fraction of the population.

Most anti-depressants target the serotonin (5-hydroxytryptamine (5-HT)) receptor, leading to pharmacological limitations such as poor treatment of refractory cases and stress-induced depression. Stress is a significant factor for development of depression and there are no pharmacological ways to mitigate stress-related depression.

Through research on a stress resilient anti-depressant model we now show that activation of 2 receptors, GABA(B)R and α2R, is sufficient to change affective state model animals.

### SUMMARY

Described are compositions, combinations, and methods of using the compositions and combinations for use in treating depression. In some embodiments, the depression is aggravated by stress. In some embodiments, the depression is stress-related or stress-induced depression. The compositions and combinations comprise a GABA B receptor (GABA(B)R) agonist and an alpha-2 adrenergic receptor (α2R) agonist.

Described are combinations, compositions, and pharmaceutical compositions comprising a GABA(B)R agonist and an α2R agonist. The GABA(B)R agonist can be an orthosteric GABA(B)R agonist, a GABA(B)R positive allosteric modulator (PAM), or a combination of an orthosteric GABA(B)R agonist and a GABA(B)R PAM. The GABA(B)R orthosteric agonist can be, but is not limited to, baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, or CGP-47656. A GABA(B)R can be, but is not limited to, CGP-7930, ADX71441, KK-92A, GS39783, BHF177, rac-BHFF, COR659, CMPPE, ORM-27669, or ASP8062. The α2R agonist can be an orthosteric α2R agonist, a α2R PAM, or a combination of an orthosteric α2R agonist and a α2R PAM. The α2R agonist can be, but is not limited to, clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, or amitraz. In some embodiments, the GABA(B)R agonist comprises baclofen and the α2R agonist comprises clonidine. The GABA(B)R agonist and the α2R agonist can be provide together or separately. In some embodiments, the GABA(B)R agonist is linked to the α2R agonist.

Described are combinations, compositions, and pharmaceutical compositions comprising GABA(B)R agonist and an α2R agonist for use in treating depression in a subject. The GABA(B)R agonist can be an orthosteric GABA(B)R agonist, a GABA(B)R positive allosteric modulator (PAM), or a combination of an orthosteric GABA(B)R agonist and a GABA(B)R PAM. The GABA(B)R orthosteric agonist can be, but is not limited to, baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, or CGP-47656. The α2R agonist can be an orthosteric α2R agonist, a α2R PAM, or a combination of an orthosteric α2R agonist and a 2R PAM. The α2R agonist can be, but is not limited to, clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, or amitraz. In some embodiments, the GABA(B)R agonist comprises baclofen and the α2R agonist comprises clonidine. The GABA(B)R agonist and the α2R agonist administered to the subject together or separately. In some embodiments, the GABA(B)R agonist is linked to the α2R agonist.

Described are methods of treating depression in a subject comprising administering the subject, a pharmaceutically effective amount of a GABA(B)R agonist and a pharmaceutically effective amount an α2R agonist. The GABA(B)R agonist and the α2R agonist can be administered to the subject together or separately. For separate administration, the GABA(B)R agonist can be administered prior to administration of the α2R agonist, after administration of the α2R agonist, or concurrently with the α2R agonist. For administration together, the GABA(B)R agonist can be linked to the α2R agonist. The GABA(B)R agonist and the α2R agonist can be administered to a subject to treat or prevent one or more symptoms associated with depression. The subject can be suffering from or diagnosed with depression. The subject can be at risk of developing depression. The depression can be, but is not limited to, stress-related or stress-induced depression.

A further understanding of the nature and advantages of the present invention may be realized by reference to the remaining portions of the specification and claims.

### BRIEF DESCRIPTION OF THE DRAWINGS

FIG. 1. Graphs illustrating TST and FST immobility time in control mice treating with control vehicle of a combination of baclofen and clonidine (ClonBac).

FIG. 2. (A) Image illustrating chronic stress assay. (B) Graph illustrating TST immobility time in control and chronically stressed mice treated with control vehicle or ClonBac. (C) Graph illustrating FST immobility time in control and chronically stressed mice treated with control vehicle or ClonBac.

FIG. 3. (A) Image illustrating unpredictable chronic mild stress (UCMS) assay. (B) Graph illustrating TST immobility time in control and UCMS mice treated with control vehicle or ClonBac. (C) Graph illustrating FST immobility time in control and UCMS mice treated with control vehicle or ClonBac. (D) Graph comparing increased FST immobility time in UCMS mice following one week of additional stress with no additional ClonBac administration.

### DETAILED DESCRIPTION

### I. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. When values are expressed as approximations, by use of the antecedent “about,” it will be understood that the particular value forms a further aspect. For example, if the value “about 10” is disclosed, then “10” is also disclosed. The term “about” or “approximately” indicates within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, and optionally one or more excipients or other material that is pharmaceutically inert. During formulation development, the excipients can be selected so that the active ingredient can reach the target site in the body at the desired rate and extent.

An “analog” refers to a molecule that structurally resembles a reference molecule (e.g., baclofen or clonidine) but which has been modified in a targeted and controlled manner, by replacing a specific substituent of the reference molecule with an alternate substituent. Compared to the reference molecule, an analog would be expected, by one skilled in the art, to exhibit the same or similar utility. Synthesis and screening of analogs to identify variants of known compounds having improved characteristics (such as higher binding affinity for a target molecule) is an approach that is well known in pharmaceutical chemistry.

A “derivative” of a first compound is a compound that has a three dimensional structure that is similar to at least a part of the first compound. In some embodiments, a derivative is a compound that is derived from, or imagined to derive from, another compound such as by substitution of one atom or group with another atom or group. In some embodiments, derivatives are compounds that at least theoretically can be formed from a common precursor compound.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to prevent or provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, e.g., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with the disease, disorder, or condition or those in which disease, disorder, or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

A “subject” can be an animal. The animal can be a mammal. A mammal can be, but is not limited to a human. The subject can have experienced and/or exhibited at least one symptom of depression or stress-induced depression. The subject can have been diagnosed with at least one symptom of depression or stress-induced depression. The subject can be experiencing or exhibiting at least one symptom of depression or stress-induced depression. The subject can be at risk of experiencing or exhibiting at least one symptom of depression or stress-induced depression In some embodiments, the at least one symptom, is caused by, has been cause by, or is likely caused by stress. The depression can be, but is not limited to, a major depressive disorder or episode.

The term “depression” includes stress-related depression, major depressive disorder, persistent depressive disorder, depression associated with bipolar disorder, seasonal affective disorder, psychotic depression, postpartum depression, premenstrual dysphoric disorder, situational depression, anhedonia, melancholy, mid-life depression, late-life depression, depression due to identifiable stressors, treatment resistant depression, or combinations thereof. In some embodiments, the depression is major depressive disorder. In some embodiments, the depression is stress-related depression.

### II. Targeting GABA(B)R and α2R

The therapeutic combinations and methods described herein are useful in the treatment of depression, including cases in which stress is an aggravating factor in the depression, such as stress-related or stress-induced depression. The concurrent agonism of GABA(B)R and α2R leads to decreased depression-related symptoms and/or behaviors.

A. GABA(B) receptor γ-Aminobutyric acid (GABA) is the major inhibitory neurotransmitter in the central nervous system (CNS) and has a key role in modulating neuronal activity. GABA mediates its action via 2 classes of receptors, ionotropic (GABA(A) and GABA(C)) and metabotropic (GABA(B)) receptors.

The G-protein coupled receptor for gamma-aminobutyric acid B receptors (GABA(B)R) are heterodimeric (GABAB1 subunit and GABAB2 subunit) metabotropic receptors. The GABA(B) receptor is a guanine nucleotide-binding (G) protein-coupled receptor that modulates calcium (Ca2+) and potassium (K⁺) channels and elicits both presynaptic and slow postsynaptic inhibition. GABA(B) receptors are broadly expressed in the nervous system, modulating synaptic excitability and plasticity in the cerebral cortex, generating rhythmic activity in cortical and thalamic circuits, relaying primary afferent input to the spinal cord and brainstem, and affecting the activity of dopaminergic and other monoaminergic neurons. These receptors have been implicated in a wide variety of neurologic and psychiatric disorders, including absence seizures, γ-hydroxybutyrate toxicity, and autoimmune limbic encephalitis.

### B. Gaba(B)R Agonists.

A GABA(B)R agonist is a compound that up-regulates or increases expression levels, or cellular activity of GABA(B)R. A GABA(B)R agonist can be an orthosteric GABA(B)R agonist, a GABA(B)R positive allosteric modulator (PAM), or a combination of an orthosteric GABA(B)R agonist and a GABA(B)R PAM.

GABA(B)R orthosteric agonists include, but are not limited to, baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, and CGP-47656.

GABA(B)R PAMs include, but are not limited to, CGP-7930, ADX71441, KK-92A, GS39783, BHF177, rac-BHFF, COR659, CMPPE, ORM-27669, and ASP8062. In some embodiments a GABA(B)R PAM can be combined with a orthosteric GABA(B)R agonist.

Baclofen (β-(4-chlorophenyl)-γ-aminobutyric acid (β-(4-chlorophenyl)-GABA); CAS No. 1134-47-0) is used to treat muscle spasticity such as from a spinal cord injury or multiple sclerosis.

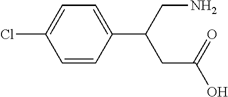 [SMILES: NCC(CC(=O)O)c1ccc(Cl)cc1]

Baclofen can be provided as, for example, a tablet, granules, a suspension, a solution (liquid), or an intrathecal solution. Baclofen can be administered once, twice, three, or four more times daily. For multiple administrations per day, the administration can be at evenly spaced intervals. In some embodiments, an effective dose of baclofen is about 5 mg, about, 10 mg, or about 20 mg. In some embodiments, baclofen is provided in a solution of about 0.05 mg/mL, about 0.5 mg/mL, about 1 mg/mL, about 2 mg/mL, about 5 mg/mL. In some embodiments, an effective dose of baclofen is about 5 mg 3-4 times per day, about 10 mg 3-4 times per day, about 15 mg 3-4 times per day, about 20 mg 3-4 times per day, or up to about 80 mg per day.

γ-Hydroxybutyrate (GHB; CAS No. 591-81-1) is used in the treatment of narcolepsy.

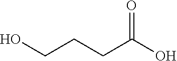

Phenibut (CAS No. 1078-21-3) is a central nervous system depressant with anxiolytic effects, and has been used to treat anxiety and insomnia.

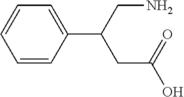 [SMILES: NCC(CC(=O)O)c1ccccc1]

4-Fluorophenibut (CAS No. 52237-19-1).

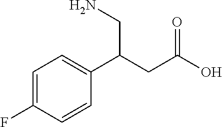 [SMILES: NCC(CC(=O)O)c1ccc(F)cc1]

### Tolibut (CAS No. 28311-38-8).

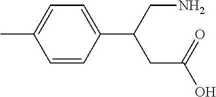

### Isovaline (CAS No. 595-39-1, 3059-97-0 (R), 595-40-4 (S)).

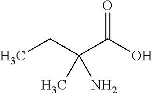

3-Aminopropylphosphinic acid (3-APPA or CGP27492; CAS No. 103680-47-3).

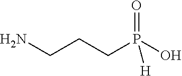

Lesogaberan (CAS No. 344413-67-8) is an experimental drug for the treatment of gastroesophageal reflux disease (fewer nervous system side effects).

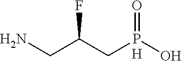

SKF-97541 (3-Aminopropyl(methyl)phosphinic acid) (CAS No. 127729-35-5).

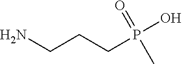

CGP-44532 (CAS No. 133345-68-3).

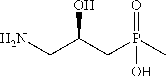

CGP-47656 (3-aminopropyl(difluoromethyl)phosphinic acid) (CAS No. 133345-73-0)

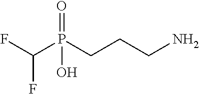

CGP-7930 (CAS No. 57717-80-3).

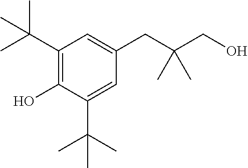 [SMILES: CC(C)(CO)Cc1cc(C(C)(C)C)c(O)c(C(C)(C)C)c1]

ADX71441 (CAS No. 1207440-88-7)

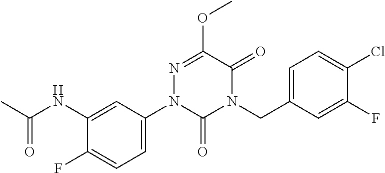 [SMILES: COc1nn(-c2ccc(F)c(NC(C)=O)c2)c(=O)n(Cc2ccc(Cl)c(F)c2)c1=O]

KK-92A ([(4-(cycloheptylamino)-5-(4-(trifluoromethyl)phenyl)pyrimidin-2-yl)methanol]).

GS39783 (N,N′-Dicyclopentyl-2-(methylthio)-5-nitro-4,6-pyrimidinediamine) (CAS No. 39069-52-8).

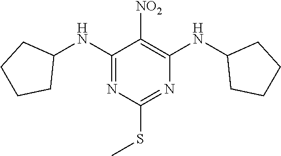 [SMILES: CSc1nc(NC2CCCC2)c([N+](=O)[O-])c(NC2CCCC2)n1]

BHF177 (N-[(1R,2R,4S)-bicyclo[2.2.1]heptan-2-yl]-2-methyl-5-[4-(trifluoromethyl)phenyl]pyrimidin-4-amine) (CAS No. 917896-43-6).

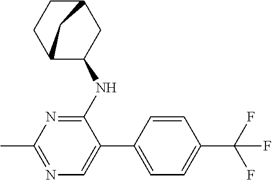

rac-BHFF (5,7-Bis(1,1-dimethylethyl)-3-hydroxy-3 (trifluoromethyl)-2 (3H)-benzofuranone) (CAS No. 123557-91-5).

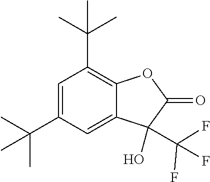 [SMILES: CC(C)(C)c1cc(C(C)(C)C)c2c(c1)C(O)(C(F)(F)F)C(=O)O2]

COR659 (CAS No. 544450-68-2).

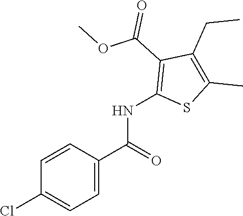 [SMILES: CCc1c(C)sc(NC(=O)c2ccc(Cl)cc2)c1C(=O)OC]

CMPPE (2-[1-[2-(4-Chlorophenyl)-5-methylpyrazol[1,5-a]pyrimidin-7-yl]-2-piperidinyl]ethanol) (CAS No. 841253-81-4).

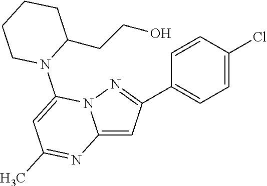 [SMILES: Cc1cc(N2CCCCC2CCO)n2nc(-c3ccc(Cl)cc3)cc2n1]

ORM-27669 ((S)-1-(5-fluoro-2,3-dihydro-1H-inden-2-yl)-4-methyl-6,7,8,9-tetrahydro-[1,2,4]triazolo[4,3-a]quinazolin-5 (4H)-one).

ASP8062.

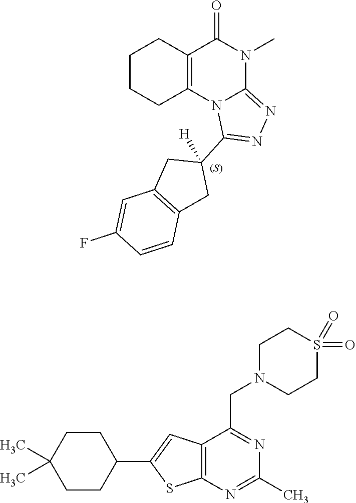

### C. Alpha-2 Adrenergic Receptor (α2R)

The alpha-2 adrenergic receptor (α2R) is a G protein-coupled receptor (GPCR) associated with the Gi heterotrimeric G-protein. It consists of three highly homologous subtypes, including α_{2A-}, α_{2B-}, and α_{2C}-adrenergic. The α2 adrenergic receptor is localized in central nervous system (CNS) structures including, but not limited to, brain, hypothalamus, hippocampus, spinal cord, cerebral cortex, and cerebellum. The α2R is located on vascular prejunctional terminals where it inhibits the release of norepinephrine (noradrenaline) in a form of negative feedback. It is also located on the vascular smooth muscle cells of certain blood vessels, such as those found in skin arterioles or on veins. Catecholamines like norepinephrine (noradrenaline) and epinephrine (adrenaline) signal through the α2-adrenergic receptor in the central and peripheral nervous system. Agonists (activators) of the α2-adrenergic receptor are frequently used in veterinary anesthesia where they affect sedation, muscle relaxation and analgesia through effects on the CNS.

### D. α2R Agonists

A α2R agonist is a compound that up-regulates or increase expression, levels, or cellular activity of α2R.

α2 receptor agonists include, but are not limited to, clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

An α2R agonist may also be a positive allosteric modulator (PAM) of α2R. In some embodiments an α2R PAM can be combined with an orthosteric α2R agonist (e.g., clonidine or fadolmidine).

Clonidine (CAS No. 4205-90-7) has been used to treat high blood pressure, attention deficit hyperactivity disorder, drug withdrawal (alcohol, opioids, or nicotine), menopausal flushing, diarrhea, spasticity, and certain pain conditions,

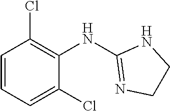 [SMILES: Clc1cccc(Cl)c1NC1=NCCN1]

Clonidine can be provided as, for example, a tablet, a transdermal patch, a topical gel, or a liquid. The liquid can be formulated for injection as epidural infusion. In some embodiments, an effective dose of clonidine is about 100 μg to about 800 μg/day. The effective dose can be administered as a divided dose. In some embodiments, an effective dose of clonidine is about 0.1 mg/day, about 0.17-0.52 mg/day, about 0.1 mg twice per day, about 0.2-0.6 mg/day, or up to about 2.4 mg/day. The effective dose can be administered as a divided dose.

Fadolmidine (CAS No. 189353-31-9)

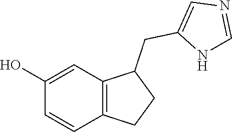 [SMILES: Oc1ccc2c(c1)C(Cc1cnc[nH]1)CC2]

Detomidine (CAS No. 76631-46-4) has been used as a sedative in horses.

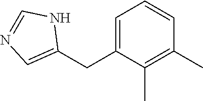

Dexmedetomidine (CAS No. 113775-47-6) has been used as an anxiolytic or sedative.

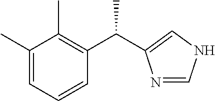

### Guanabenz (CAS No. 5051-62-7).

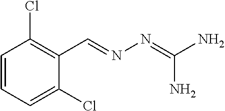 [SMILES: NC(N)=N/N=C/c1c(Cl)cccc1Cl]

Guanoxabenz (CAS No. 24047-25-4 check) is a metabolite of guanabenz.

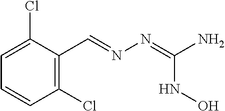 [SMILES: N/C(=N/N=C/c1c(Cl)cccc1Cl)NO]

### Guanethidine (CAS No. 645-43-2).

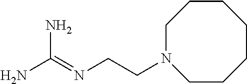 [SMILES: NC(N)=NCCN1CCCCCCC1]

Guanfacine (CAS No. 29110-47-2) has been used to treat ADHD and high blood pressure.

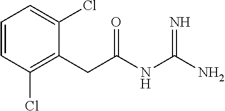 [SMILES: N=C(N)NC(=O)Cc1c(Cl)cccc1Cl]

Lofexidine (CAS No. 31036-80-3) has been used to treat hypertension and opioid withdrawal.

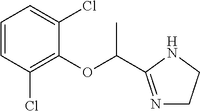 [SMILES: CC(Oc1c(Cl)cccc1Cl)C1=NCCN1]

Medetomidine (CAS No. 86347-14-0) has been used as an anesthetic or analgesic in veterinary medicine.

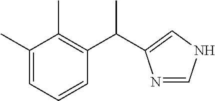

Methyldopa (CAS No. 41372 Aug. 1) has been used to treat high blood pressure.

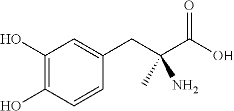

Methylnorepinephrine (CAS No. 829-74-3) has been used as a decongestant or vasoconstrictor.

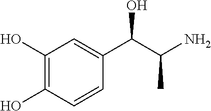

Norepinephrine (CAS No. 51-41-2) has been used to treat critically low blood pressure.

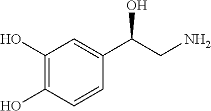 [SMILES: NC[C@H](O)c1ccc(O)c(O)c1]

Tizanidine (CAS No. 51322-75-9) has been used to treat muscle spasticity.

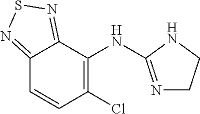 [SMILES: Clc1ccc2nsnc2c1NC1=NCCN1]

Xylazine (CAS No. 7361-61-7) has been use in sedation or as an anesthetic or analgesic in veterinary medicine.

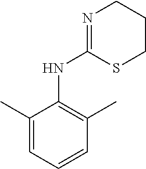

(R)-3-nitrobiphenyline (CAS No. 945618-95-1).

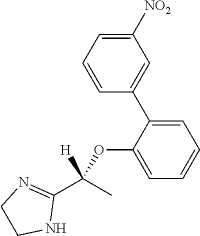 [SMILES: C[C@@H](Oc1ccccc1-c1cccc([N+](=O)[O-])c1)C1=NCCN1]

Brimonidine (CAS No. 59803-98-4) has been used to treat ocular hypertension.

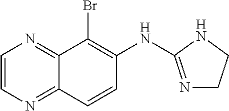 [SMILES: Brc1c(NC2=NCCN2)ccc2nccnc12]

### Amitraz (CAS No. 33089-61-1).

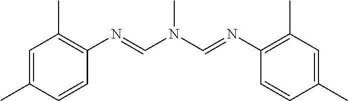

In some embodiments, the GABA(B)R agonist comprises baclofen and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises GHB and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises γ-aminobutyric acid and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises phenibut and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises 4-Fluorophenibut and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises tolibut and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises isovaline and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises 3-aminopropyl-phosphinic acid and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises lesogaberan and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises SKF-97541 and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises CGP-44532 and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the GABA(B)R agonist comprises CGP-47656 and the α2R agonist comprises a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz.

In some embodiments, the α2R agonist comprises clonidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises fadolmidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises detomidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises dexmedetomidine and the GABA(B)R agonist comprises a therapeutic selected from the group consisting of: baclofen, 7-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises guanabenz and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises guanoxabenz and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises guanethidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises guanfacine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises lofexidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises medetomidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises methyldopa and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises methylnorepinephrine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises norepinephrine and the GABA(B)R agonist comprises a therapeutic selected from the group consisting of: baclofen, 7-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises tizanidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises xylazine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises (R)-3-nitrobiphenyline and the GABA(B)R agonist comprises a therapeutic selected from the group consisting of: baclofen, 7-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises brimonidine and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the α2R agonist comprises amitraz and the GABA(B)R agonist comprises a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062.

In some embodiments, the GABA(B)R agonist comprises baclofen and the α2R agonist comprises clonidine.

In some embodiments, the GABA(B)R agonist comprises GHB and the α2R agonist clonidine.

In some embodiments, the GABA(B)R agonist comprises γ-aminobutyric acid and the α2R agonist clonidine.

In some embodiments, the GABA(B)R agonist comprises phenibut and the x2R agonist comprises clonidine.

In some embodiments, the GABA(B)R agonist comprises 4-fluorophenibut and the α2R agonist comprises clonidine.

In some embodiments, the GABA(B)R agonist comprises tolibut and the α2R agonist comprises clonidine.

In some embodiments, the GABA(B)R agonist comprises isovaline and the α2R agonist comprises clonidine.

In some embodiments, the GABA(B)R agonist comprises 3-aminopropyl-phosphinic acid and the α2R agonist comprises clonidine.

In some embodiments, the GABA(B)R agonist comprises lesogaberan and the α2R agonist comprises clonidine.

In some embodiments, the GABA(B)R agonist comprises SKF-97541 and the α2R agonist comprises clonidine.

In some embodiments, the GABA(B)R agonist comprises CGP-44532 and the α2R agonist comprises clonidine.

In some embodiments, the GABA(B)R agonist comprises CGP-47656 and the α2R agonist comprises clonidine.

In some embodiments, the GABA(B)R agonist comprises baclofen and the α2R agonist comprises fadolmidine.

In some embodiments, the GABA(B)R agonist comprises baclofen and the α2R agonist comprises detomidine.

In some embodiments, the GABA(B)R agonist comprises baclofen and the α2R agonist comprises dexmedetomidine.

In some embodiments, the GABA(B)R agonist comprises baclofen and the α2R agonist comprises guanabenz.

In some embodiments, the GABA(B)R agonist comprises baclofen and the α2R agonist comprises guanoxabenz.

In some embodiments, the GABA(B)R agonist comprises baclofen and the α2R agonist comprises guanethidine.

In some embodiments, the GABA(B)R agonist comprises baclofen and the α2R agonist comprises guanfacine.

In some embodiments, the GABA(B)R agonist comprises baclofen and the α2R agonist comprises lofexidine.

In some embodiments, the GABA(B)R agonist comprises baclofen and the x2R agonist comprises medetomidine.

In some embodiments, the GABA(B)R agonist comprises baclofen and the α2R agonist comprises methyldopa.

In some embodiments, the GABA(B)R agonist comprises baclofen and the α2R agonist comprises methylnorepinephrine.

In some embodiments, the GABA(B)R agonist comprises baclofen and the α2R agonist comprises norepinephrine.

In some embodiments, the GABA(B)R agonist comprises baclofen and the α2R agonist comprises tizanidine.

In some embodiments, the GABA(B)R agonist comprises baclofen and the α2R agonist comprises xylazine.

In some embodiments, the GABA(B)R agonist comprises baclofen and the α2R agonist comprises (R)-3-nitrobiphenyline.

In some embodiments, the GABA(B)R agonist comprises baclofen and the α2R agonist comprises brimonidine.

In some embodiments, the GABA(B)R agonist comprises baclofen and the α2R agonist comprises amitraz.

### III. Pharmaceutical Compositions

In some embodiments, a GABA(B)R agonist, an α2R agonist, a combination of a GABA(B)R agonist and a α2R agonist, or a cross-linked GABA(B)R/α2R agonist is formulated with one or more pharmaceutically acceptable excipients (including vehicles, carriers, diluents, and/or delivery polymers), thereby forming a pharmaceutical composition or medicament suitable for in vivo delivery to a subject, such as a human.

A pharmaceutical composition or medicament includes a pharmacologically effective amount of the active compound and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support, or enhance stability, bioavailability or patient acceptability of the API, c) assist in product identification, and/or d) enhance any other attribute of the overall safety and effectiveness of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants, such as preservatives, wetting agents, emulsifying agents, and dispersing agents. A carrier may also contain isotonic agents, such as sugars, polyalcohols, sodium chloride, and the like into the compositions.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The phrase “pharmaceutically acceptable” indicates that the substance or composition must be compatible chemically and/or toxicologically, with the other ingredients comprising a formulation, and/or the mammal being treated therewith. Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals, and more particularly in humans.

A pharmaceutical composition containing a GABA(B)R agonist, an α2R agonist, or both a GABA(B)R agonist and an α2R agonist (unlinked or crosslinked) may further comprise or be used in combination with one or more additional active ingredients. The additional active pharmaceutical ingredients can be, but are not limited to, an analgesic, an antipsychotic, a mood stabilizer, or an antidepressant.

### IV. Formulation.

The GABA(B)R agonist and the α2R agonist are formulated for administration in vivo. The GABA(B)R agonist and an α2R agonist can be formulated together or for administration separately. In some embodiments, the GABA(B)R agonist and an α2R agonist are formulated together. In some embodiments, the GABA(B)R agonist comprises baclofen and the α2R agonist comprises clonidine.

In some embodiments, a GABA(B)R agonist and an α2R agonist are linked to form a crosslinked GABA(B)R/α2R agonist. The linkage can be a covalent linkage or a non-covalent linkage. In some embodiments, the GABA(B)R agonist comprises baclofen and is linked to an α2R agonist comprising a therapeutic selected from the group consisting of: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz. In some embodiments, the α2R agonist comprises clonidine and is linked to a GABA(B)R agonist comprising a therapeutic selected from the group consisting of: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062. In some embodiments, the GABA(B)R agonist comprises baclofen and the α2R agonist comprises clonidine and the baclofen is linked to the clonidine.

Any of the described GABA(B)R agonists, α2R agonists, combinations of a GABA(B)R agonist and a α2R agonist, crosslinked GABA(B)R/α2R agonists, or pharmaceutical compositions can be formulated or manufactured as a solid, a powder (e.g., a powder or lyophilized formulation), a tablet (e.g., a pill), a capsule (e.g., a time-release capsule), granules, a suspension in an aqueous liquid or non-aqueous liquid, an emulsion, or a liquid. In some embodiments, the GABA(B)R agonist is formulated or manufactured as a solid, a powder (e.g., a powder or lyophilized formulation), a tablet (e.g., a pill), a capsule (e.g., a time-release capsule), granules, a suspension in an aqueous liquid or non-aqueous liquid, an emulsion, or a liquid. In some embodiments, the α2R agonist is formulated or manufactured as a solid, a powder (e.g., a powder or lyophilized formulation), a tablet (e.g., a pill), a capsule (e.g., a time-release capsule), granules, a suspension in an aqueous liquid or non-aqueous liquid, an emulsion, or a liquid. In some embodiments, the GABA(B)R agonist and α2R agonist are formulated or manufactured together as a solid, a powder (e.g., a powder or lyophilized formulation), a tablet (e.g., a pill), a capsule (e.g., a time-release capsule), granules, a suspension in an aqueous liquid or non-aqueous liquid, an emulsion, or a liquid. In some embodiments, the crosslinked GABA(B)R/α2R agonist is formulated or manufactured as a solid, a powder (e.g., a powder or lyophilized formulation), a tablet (e.g., a pill), a capsule (e.g., a time-release capsule), granules, a suspension in an aqueous liquid or non-aqueous liquid, an emulsion, or a liquid.

The GABA(B)R agonist and an α2R agonist can be formulated together or separately for oral administration, parenteral administration, intravenous (IV) administration, intrathecal administration, injection, infusion, intradermal administration, subcutaneous administration, intramuscular administration, intraperitoneal administration, aerosol or inhalation administration, nasal administration, topical administration, rectal administration, transmucosal administration, or transdermal administration. The crosslinked GABA(B)R/α2R agonist can be formulated for oral administration, parenteral administration, intravenous (IV) administration, intrathecal administration, injection, infusion, intradermal administration, subcutaneous administration, intramuscular administration, intraperitoneal administration, aerosol or inhalation administration, nasal administration, topical administration, rectal administration, transmucosal administration, or transdermal administration.

Any of the compounds or pharmaceutical compositions identified herein for use in the described methods are administered to a subject in an amount that is sufficient to achieve a desired therapeutic effect in the subject. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors, including the activity of the particular GABA(B)R agonist, α2R agonist, or crosslinked GABA(B)R/α2R agonist employed, or the ester, salt or amide thereof, the route of administration, the time of administration, or the rate of excretion of the particular compound(s) being employed. Dosage can also depend on the duration of the treatment, or other drugs, compounds, and/or materials used in combination with the employed GABA(B)R agonist, α2R agonist, or crosslinked GABA(B)R/α2R agonist. Age, gender, weight, condition, general health, and prior medical history of the subject being treated can also affect dosage. Methods for determining optimal dosages are described in the art, e.g., Remington: The Science and Practice of Pharmacy, Mack Publishing Co., 20th ed., 2000. In some embodiments, the GABA(B)R agonist and/or the α2R agonist are administered to a subject at their recognized dosage levels for treatment of a subject.

### V. Kits

Also described herein are kits for administering one or more GABA(B)R agonists, α2R agonists, combinations of a GABA(B)R agonist and an α2R agonist, or crosslinked GABA(B)R/α2R agonists, or formulations thereof, or pharmaceutical compositions described herein to a subject for the treatment of depression. In some embodiments, a kit can contain one or more of the described GABA(B)R agonists, α2R agonists, combinations of a GABA(B)R agonist and an α2R agonist, crosslinked GABA(B)R/α2R agonists, or pharmaceutical compositions containing a GABA(B)R agonist, a α2R agonist, a combination of a GABA(B)R agonist and an α2R agonist, or a crosslinked GABA(B)R/α2R agonist. In some embodiments, a kit comprises a GABA(B)R agonist and a α2R agonist, or crosslinked GABA(B)R/α2R agonist, or a pharmaceutical composition containing GABA(B)R agonist and a α2R agonist, or crosslinked GABA(B)R/α2R agonist.

In some embodiments, a kit comprises one or more dosage units comprising an effective amount of one or more compounds described herein for administration to a subject at a predetermined frequency. The dosage unit may be formulated for delivery by any means. In certain embodiments, the dosage unit is formulated for oral, parenteral, intravenous, intranasal, intramuscular, sublingual, transdermal, or rectal delivery. In certain embodiments, the dosage unit is formulated for oral delivery.

In some embodiments, a kit further comprises one or more additional components. The one or more additional components can be, but are not limited to, at least one additional active pharmaceutical ingredient, one or more inactive ingredients, an excipient, a diluent, and the like.

In some embodiments, a kit further comprises optional components that aid in the administration of the unit dose to a subject, including but not limited to: vials for reconstituting powder forms, syringes for injection, customized IV delivery systems, inhalers, etc.

A kit can be manufactured as a single use unit dose for one subject, multiple dose units for a particular subject (at a constant dose or in which the individual compounds may vary in potency as therapy progresses), or multiple dose units suitable for administration to multiple subjects (“bulk packaging”). The kit components may be assembled in cartons, blister packs, bottles, tubes, and the like.

In some embodiments, a kit further comprises instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration. The instructions may include direction for preparation and/or administration of the dosage form by the patient or person administering the drug to the patient. The instructions may include a depression symptom rating scale questionnaire. The questionnaire may be for use by the patient alone or in combination with a physician. The questionnaire may be useful for determining the level of depression of the patient at any stage of compound administration.

### VI. Methods of Treating Depression

Methods of treating a subject suffering from depression, diagnosed with depression, or at risk of developing depression, including stress-related depression or stress-induced depression, are provided. Methods of treating one or more symptoms associated with depression, stress-related depression or stress-induced depression are also described. The methods comprise administering to a subject in need of such treatment a combination of an effective amount of a GABA(B)R agonist and an effective amount of an α2R agonist. The GABA(B)R agonist and the α2R agonist can be administered to the subject together or separately. In some embodiments, the GABA(B)R agonist and the α2R agonist are covalently linked to form a crosslinked GABA(B)R/α2R agonist. For separate administration, the GABA(B)R agonist can be administered prior to administration of the α2R agonist, subsequent to administration of the α2R agonist or currently with administration of the α2R agonist. The GABA(B)R agonist and the α2R agonist can be administered to the subject as a first pharmaceutical composition containing the GABA(B)R agonist and a second pharmaceutical composition containing the α2R agonist, as a pharmaceutical composition containing both the GABA(B)R agonist and the α2R agonist, or a pharmaceutical composition containing a crosslinked GABA(B)R/α2R agonist. The GABA(B)R agonist can be, but is not limited to: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, and ASP8062. The α2R agonist can be, but is not limited to: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, and amitraz. In some embodiments, the GABA9B)R agonist is baclofen and the α2R agonist is clonidine. In some embodiments, the baclofen and the clonidine are crosslinked. The subject can be, but is not limited to, a human. Any of the described pharmaceutical compositions or formulations may be used in the methods of treatment.

A pharmaceutical composition containing a GABA(B)R agonist, an α2R agonist, a combination of a GABA(B)R agonist and a α2R agonist, or a cross-linked GABA(B)R/α2R agonist and optionally another therapeutic agent can be administered by a variety of methods known in the art. The routes and/or modes of administration may vary depending upon the desired results. Depending on the route of administration, an active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

In some embodiments, the GABA(B)R agonist and an α2R agonist administered together orally. In some embodiments, the GABA(B)R agonist and an α2R agonist administered together parenterally.

In some embodiments, the GABA(B)R agonist and α2R agonist are administered to a subject at dosage levels recognized or recommended for treating other conditions.

In some embodiments, the GABA(B)R agonist and α2R agonist are administered according to their recognized administration routes.

The compounds and pharmaceutical compositions disclosed herein can be administered to a subject once per day, more than once a day, for example, 2, 3, 4, 5, or 6 times a day, or as needed.

Treating one or more symptoms associated with a depression, included stress-related depression or stress-induced depression includes, but is not limited to, decreasing or suppressing the one or more symptoms, decreasing the frequency of the one or more symptoms, decreasing the severity of the one or more symptoms, delaying or avoiding of the development of additional symptoms, or decreasing progression of the one or more symptoms. Symptoms associated with stress-related depression or stress-induced depression include, but are not limited to, feelings of agitation, frustration, anger, or loss of control, moodiness, difficulty relaxing or sleeping, low self-esteem, loneliness, social isolation, low energy, headaches, upset stomach, diarrhea, constipation, nausea, muscle tension, frequent illness, loss of sexual desire and/or ability, nervousness, shaking, ringing in the ear, cold or sweaty hands and feet, dry mouth, difficulty swallowing, jaw clenching, teeth grinding, worry, forgetfulness, disorganization, inability to focus, problems with memory or retention, poor judgment, pessimism, changes in appetite, procrastination, avoiding responsibilities, increased alcohol or drug use, nervous behavior, nail biting, fidgeting, and pacing. Treating the stress-related depression or stress-induced depression can also include improving the quality of life of the person suffering from the depression of decreasing the dosage of one or more other therapeutics taken by the subject to treat depression.

In some embodiments, the method further comprises administering to the subject one or more additional active ingredients. The one or more additional active ingredients may be administered simultaneously, separately, or sequentially. The one or more additional active ingredients may be combined with a pharmaceutical composition containing a GABA(B)R agonist, a pharmaceutical composition containing an α2R agonist, or a pharmaceutical composition containing both a GABA(B)R agonist and an α2R agonist. The one or more additional active ingredients may be, but is not limited to, an antidepressant. The anti-depressant may be an anti-depressant that targets the serotonin receptor. The one or more additional active ingredients may be, but are not limited to a N-methyl-D-aspartate receptor antagonist, a norepinephrine reuptake inhibitor, a selective serotonin reuptake inhibitor (SSRI), a monoamine oxidase inhibitor (MAOI), a reversible inhibitor of monoamine oxidase (RIMA), a serotonin and noradrenaline reuptake inhibitor (SNRI), a noradrenergic and specific serotonergic antidepressant (NaSSA), or a corticotropin releasing factor (CRF) antagonist, or an atypical antidepressant. An N-methyl-D-aspartate receptor antagonist can be, but is not limited to: ketamine, esketamine, or arketamine. A norepinephrine reuptake inhibitor can be, but is not limited to amitriptyline, clomipramine, doxepin, imipramine, trimipramine, amoxapine, desipramine, maprotiline, nortriptyline, protriptyline, or reboxetine. A SSRI can be, but is not limited to, fluoxetine, fluvoxamine, paroxetine, or sertraline. A MAOI can be, but is not limited to, isocarboxazid, phenelzine, tranylcypromine, or selegiline. An RIMA can be, but is not limited to, moclobemide. A SNRI can be, but is not limited to, venlafaxine. An atypical antidepressant includes, but is not limited to, bupropion, lithium, nefazodone, trazodone, viloxazine, or sibutramine. Additional antidepressants can be, but are not limited to, adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine, or zometapine.

The subject can be any subject (a) that has experienced or exhibited at least one symptom of depression or stress-related or stress-induced depression; (b) that has not exhibited any symptoms of depression or stress-related or stress-induced depression, but who has been deemed, such as by a physician, clinician, or other medical profession, to be at risk of developing symptoms of depression of stress-related or stress-induced depression; (c) that has previously experienced or exhibited at least one symptom of depression or stress-related or stress-induced depression and deemed at risk of having new episodes of depression.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### Examples

### Example 1. Effect of the Combination of Combination GABA(B)R Agonist and an α2R Agonist Therapy on Naïve Mice Behavior

Behavior of naïve (non-stressed) mice was analyzed using a tail suspension test (TST) or forced swim test (FST). TST ad FST are often used to measure the effectiveness of anti-depressants.

Mice were handled and injected for 2 days prior to behavioral test. Mice were placed back into their home cage after each injection. Mice were randomly chosen to be injected with either vehicle or ClonBac. A final concentration of Clonidine (0.5 mg/kg) and Baclofen (0.5 mg/kg) were dissolved in DMSO. The combination of clonidine and baclofen is referred to as ClonBac.

TST was performed 30 min after injection time. To perform the TST, the tails of the mice were wrapped with tape that covered approximately 4/5 of the tail length and then fixed upside down on a hook. The immobility time of each mouse was recorded and tracked over a 6 min period. The Porsolt FST was conducted using vertical clear glass cylinder (10 cm in diameter, 25 cm in height) filled with water (25° C.). The mice spent 6 min in the water and immobility was scored from 2 to 6 min. A mouse was regarded as immobile when floating motionless or making only those movements necessary to keep its head above the water.

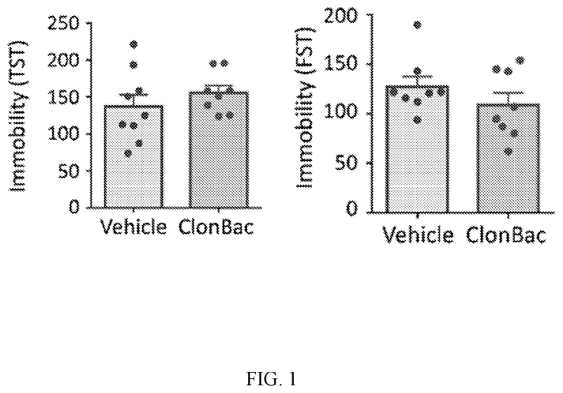

Results: No difference in immobility was found between vehicle and ClonBac treated mice in the TST or FST (FIG. 1). Indicated that for naïve mice, agonism of GABA(B) and α2 receptors does not affect depression.

### Example 2. Effect of ClonBac Combination Therapy on Stressed Mice Behavior

This study investigated the effects of ClonBac in a mouse model of chronic stress. Physical restraint stress (PRS) was used to induce stress in the mice. Behavior of stressed mice was analyzed using a tail suspension test (TST) and forced swim test (FST). Specifically, the effect of administration of ClonBac on depressive-like behaviors seen in mice subjected PRS was analyzed/This model is widely used as a preclinical animal model of depression.

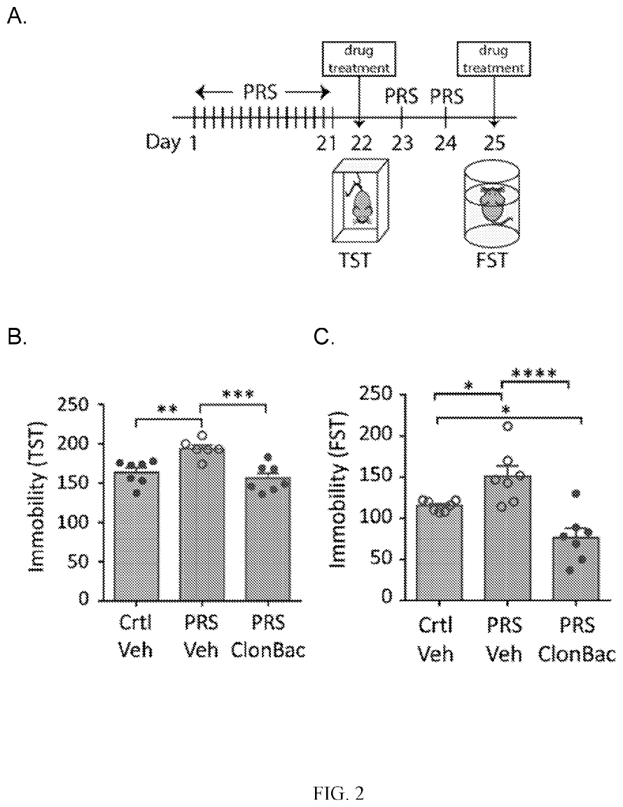

Mice were randomly selected to be in the chronically stress (PRS group) or non-stressed group. For the PRS mice, mice were housed individually and subjected to daily physical restraint (3 hr) for 3 weeks (days 1-21). Two days prior to behavioral testing all mice were weighed and injected with vehicle (intraperitoneal). On day 22, mice were injected with ClonBac or vehicle. 30 min after the injection, the mice were subjected to TST. To negate any potential acute stress on the control group mice were left in their home cage for 2 days, while the stressed mice underwent PRS. On day 25 mice were injected with ClonBac or vehicle. 30 min after injection, the mice were subjected to FST (FIG. 2A).

Results: Mice exposed to PRS (PRS+Veh) exhibited higher immobility times in the TST compared to control mice, indicating a depressive-like phenotype. ClonBac treatment attenuated the immobility TST time in PRS mice, similar to unstressed control mice (FIG. 2B). In the FST, PRS+Veh exposed mice showed a higher immobility time compared to control mice, whereas ClonBac treatment mice showed a lower immobility time (FIG. 2C). Administration of ClonBac ameliorated depressive-like behaviors in a chronic stress model of depression indicated an antidepressant-like effect.

### Example 3. Effect of ClonBac Combination Therapy on Stressed Mice Behavior

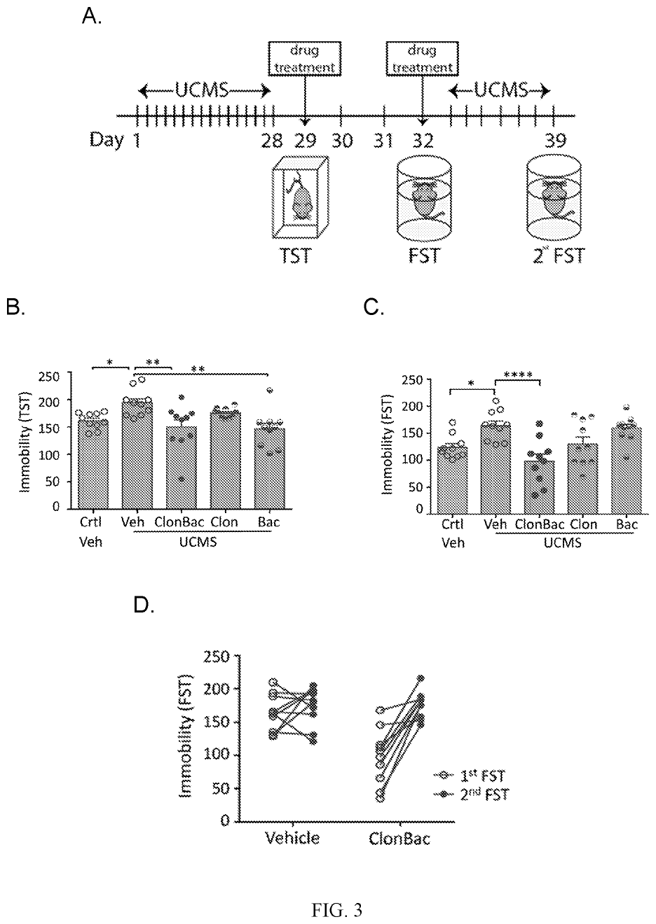

This study investigated the effects of ClonBac in a mouse model of unpredictable chronic mild stress (0). For the UCMS paradigm, mice were housed in individual cages and were subjected to 4 weeks of stressors, with two stressors applied within a 24 hr period. Stressors were applied pseudo-randomly and included: intermittent bell (10 db, 1/10 s), continuous white noise for 4 h, rat odor, 45° cage tilt for 4 h, 30 minute cage shaking, soiled bedding for 8 hr, paired housing with a new partner for 2 h, overnight illumination, removal of nesting material for 12 hr, placement of a novel object in home cage for 3 h, and physical restraint for 1 h. Stressors continued to be applied during the behavioral testing phase. This model is widely used as a preclinical animal model of depression. No stressors were applied on the day of testing (18 h prior to any behavioral testing) to avoid effects of fatigue and acute stress. Non-stressed mice were left undisturbed in their home cages. Two days prior to behavioral testing all mice were weighed and injected with vehicle (intraperitoneal). On day 29, mice were injected with vehicle of ClonBac and a TST was administered (30 min after injection). One day 31, mice were injected with vehicle or ClonBac and a FST was administered (30 min after injection). UCSM was applied on days 32-39 and a second FST was administered on day 39 (FIG. 3A).

Results: Mice subjected to UCMS showed an increased immobility time on the TST. Treatment with ClonBac or baclofen significantly attenuated immobility TST time in UMCS mice (FIG. 3B). Similarly, a higher immobility time was exhibited in mice subjected to UMCS compared to control mice in the FST. UCMS mice injected with ClonBac showed a lower immobility time in the FST compared to vehicle treated UCMS mice or either clonidine or baclofen alone (FIG. 3C).

A second FST on UCMS mice was conducted seven days after the initial test to determine if ClonBac effects were long lasting. ClonBac treated mice showed similar immobility times compared to vehicle treated suggesting that Clonbac did not have a long lasting antidepressant-like effect on UCMS (FIG. 3D).

Administration of ClonBac ameliorated depressive-like behaviors in the UCMS model of depression indicated an antidepressant effect.

1. A pharmaceutical combination comprising a GABA(B) receptor (GABA(B)R) agonist and an α2 receptor (α2R) agonist.

2. The pharmaceutical combination of claim 1, wherein the GABA(B)R agonist comprises:
baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, or ASP8062, or a combination thereof.

3. The pharmaceutical combination of claim 2, wherein the GABA(B)R agonist comprises baclofen.

4. The pharmaceutical combination of any one of claims 1-3, wherein the α2R agonist comprises: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, or amitraz.

5. The pharmaceutical combination of claim 4, wherein the α2R agonist comprises clonidine.

6. The pharmaceutical combination of claim 1, wherein the α2R agonist comprises clonidine and the GABA(B)R comprises: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, or ASP8062, or a combination thereof.

7. The pharmaceutical combination of any one of claims 1-6, wherein the GABA(B)R agonist comprises baclofen and the α2R agonist comprises clonidine.

8. The pharmaceutical combination of any one of claims 1-7, wherein the GABA(B)R agonist and the α2R agonist are provided together in a pharmaceutical composition.

9. The pharmaceutical combination of claim 8, wherein the GABA(B)R agonist is linked to the α2R agonist.

10. The pharmaceutical combination of claim 9, wherein the GABA(B)R agonist comprises baclofen and the α2R agonist comprises clonidine.

11. The pharmaceutical combination of any one of claims 1-10, for use in treating depression.

12. The pharmaceutical combination of any one of claims 1-10, for use in treating one or more symptoms of depression.

13. The pharmaceutical combination of claim 11 or 12, wherein the depression comprises stress-related or stress-induced depression.

14. A method of treating a depression in a subject comprising administering to the subject in need of such treatment a pharmaceutically effective amount the pharmaceutical combination of any one of claims 1-10.

15. A method of treating depression in a subject comprising administering to the subject in need of such treatment a combination of a pharmaceutically effective amount of a GABA(B)R agonist and a pharmaceutically effective amount an α2R agonist.

16. The method of claim 15, wherein the GABA(B)R agonist comprises: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, or ASP8062, or a combination thereof.

17. The method of claim 16, wherein the GABA(B)R agonist comprises baclofen.

18. The method of any one of claims 15-17, wherein the α2R agonist comprises: clonidine, fadolmidine, detomidine, dexmedetomidine, guanabenz, guanoxabenz, guanethidine, guanfacine, lofexidine, medetomidine, methyldopa, methylnorepinephrine, norepinephrine, tizanidine, xylazine, (R)-3-nitrobiphenyline, brimonidine, or amitraz.

19. The method of claim 18, wherein the α2R agonist comprises clonidine.

20. The method of claim 15, wherein the α2R agonist comprises clonidine and the GABA(B)R comprises: baclofen, γ-hydroxybutyrate (GHB), γ-aminobutyric acid, phenibut, 4-Fluorophenibut, tolibut, isovaline, 3-aminopropyl-phosphinic acid, lesogaberan, SKF-97541, CGP-44532, CGP-47656, CGP-7930, ADX71441, KK-92A, GS39783, BHF177, rac-BHFF, COR659, CMPPE, ORM-27669, or ASP8062, or a combination thereof.

21. The method of any one of claims 15-20, wherein the GABA(B)R agonist comprises baclofen and the α2R agonist comprises clonidine.

22. The method of any one of claims 15-21, wherein the GABA(B)R agonist and the α2R agonist are provided together in a pharmaceutical composition.

23. The method of claim 22, wherein the GABA(B)R agonist is linked to the α2R agonist.

24. The method of claim 23, wherein the GABA(B)R agonist comprises baclofen and the α2R agonist comprises clonidine.

25. The method of any one of claims 15-24, wherein the subject (a) has been diagnosed with depression; (b) has been diagnosed with stress-related depression; (c) is at risk of developing depression; or (d) is at risk of developing stress-related depression.

26. The method of any one of claims 15-25, wherein treating depression comprises treating or preventing at least one symptom of depression.

27. A compound comprising a GABA(B)R agonist linked to an α2R agonist.

28. The compound of claim 27, wherein the GABA(B)R agonist is covalently linked to the α2 receptor (α2R) agonist.

29. The compound of claim 28, wherein the GABA(B)R agonist comprises baclofen or a derivative thereof, the α2R agonist comprises clonidine or a derivative thereof, or the GABA(B)R agonist comprises baclofen or a derivative thereof and the α2R agonist comprises clonidine or a derivative thereof.

30. A kit comprising the pharmaceutical combination of any one of claims 1-10.
